(19) United States
(12) Patent Application Publication
(10) Pub. No.: US 2012/0184868 A1
(43) Pub. Date: Jul. 19, 2012

(54) APPARATUS AND METHOD OF ENHANCING MEMORY ABILITY AND PARASYMPATHETIC ACTIVITY

(75) Inventor: Fu-Zen Shaw, Tainan City (TW)

(73) Assignee: NATIONAL CHENG KUNG UNIVERSITY, Tainan City (TW)

(21) Appl. No.: 13/183,841

(22) Filed: Jul. 15, 2011

(30) Foreign Application Priority Data
Jan. 14, 2011 (TW) 100101535

(51) Int. Cl. G09B 19/00; A61B 5/048

(52) U.S. Cl. 600/544; 434/236

(57) ABSTRACT

An apparatus and a method of enhancing at least one of memory ability and parasympathetic activity of a subject are provided. The apparatus includes a training element for increasing a mu rhythm of the subject, and the method includes a step of increasing the mu rhythm of the subject.

### FIELD OF THE INVENTION

The present invention relates to an apparatus of enhancing memory ability and parasympathetic activity and a method thereof, and more particularly to a neurofeedback training device and a method of enhancing memory ability and parasympathetic activity.

### BACKGROUND OF THE INVENTION

Recently, there are many researches discussing the effect, resulted from brain signals of different frequency bands, upon various diseases and cognitive functions. It has been found that different brain rhythms may affect different cognitive functions in a brain, wherein the sensorimotor rhythm (SMR) has been found to be relative to attention system.

SMR is first found in a cat. When a tension of a muscle of a cat reduces, a regular brain wave of 10-20 Hz is found in the somatosensory cortex of the brain of the cat. Recently, the generation of SMR in a human being has been further studied and understood. When a human is in a relaxed state, a certain SMR rhythm indeed exists in the somatosensory cortex of the brain. In fact, there are two kinds of SMR having different occurrence locations and functions in a human being. One is high frequency SMR (15-20 Hz), which occurs in the motor cortex in front of central sulcus and has a function relating to the generation and the end of an action. The other one is low frequency SMR (8-12 Hz), which occurs in the somatosensory cortex in back of central sulcus and is also called mu rhythm (μ rhythm). When the activity of μ rhythm occurs in the somatosensory cortex, the increase of the energy about 10 Hz can be clearly found, which is accompanied with a harmonic wave of 20 Hz at times. In this situation, the action of the human is in a completely stationary state, and the activity of μ rhythm will suddenly decrease when a somatosensory stimulation is received or a motion is generated.

Neurofeedback training is a therapy by feeding back the activity of the brain wave of a subject via the stimulations such as the senses of sight, hearing and touch. Such a therapy has been generally utilized in treating neurological disorders and mental diseases, or improving cognitive functions, and so on. Currently, most of the neurofeedback training systems focus on the mentioned high frequency SMR, or on the trainings of other frequency bands of the brain wave, e.g. α and θ rhythms. As to the application of μ rhythm, the energy strength thereof may be used to control a computer, a machine or other devices, wherein the effect of inhibiting μ rhythm is selected to be a modulation for a machine or a human-machine interface.

Based on the above, researches and applications of brain signals from different frequency bands of the brain wave will benefit humans in improving certain diseases or cognitive functions. Though positive effects of many neurofeedback training methods of the prior arts on normal humans or patients have been proved, the causes thereof are hard to be explained. Particularly, in the prior arts, the training of SMR and the change of cognitive functions still have controversial issues. That is, the relationship between the training and the improved result of the condition is unclear until now. Accordingly, there is a need to develop and verify a novel neurofeedback training device and a method thereof.

### SUMMARY OF THE INVENTION

In accordance with one aspect of the present invention, an apparatus of enhancing at least one of a memory ability and a parasympathetic activity of a subject is provided. The apparatus includes a training element for increasing a mu rhythm of the subject.

In accordance with another aspect of the present invention, a method of enhancing a memory ability of a subject is provided. The method includes a step of increasing a mu rhythm of the subject.

In accordance with a further aspect of the present invention, a method of enhancing a parasympathetic activity of a subject is provided. The method includes a step of increasing a mu rhythm of the subject.

The above objects and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 is a diagram showing an embodiment of the apparatus of enhancing at least one of a memory ability and a parasympathetic activity of the present invention.

FIG. 2 is a diagram showing another embodiment of the apparatus of enhancing at least one of a memory ability and a parasympathetic activity of the present invention.

FIG. 3(A) is a flow chart showing an embodiment of a method of enhancing a memory ability of the present invention.

FIG. 3(B) is a flow chart showing an embodiment of a method of enhancing a parasympathetic activity of the present invention.

FIGS. 4(A)-4(C) are diagrams showing the ways of recording brain wave and displaying training interface in the present invention.

FIGS. 5(A)-5(F) are diagrams showing change of energy of Control group, SMR group and Mu group, respectively, during the neurofeedback training of the present invention.

FIGS. 6(A)-6(F) are cumulative length diagrams for the occurred signals of the respective groups.

FIGS. 7(A)-7(E) are contrast diagrams showing varient amounts of the analysis of heart rate variability for the respective groups.

FIGS. 8(A)-8(F) are contrast diagrams showing the accuracy and difference value of the evaluation of cognitive ability for the respective groups.

### DETAILED DESCRIPTION OF THE PREFERRED EMBODIMENT

The present invention will now be described more specifically with reference to the following embodiments. It is to be noted that the following descriptions of embodiments of this invention are presented herein for the purposes of illustration and description only; it is not intended to be exhaustive or to be limited to the precise form disclosed.

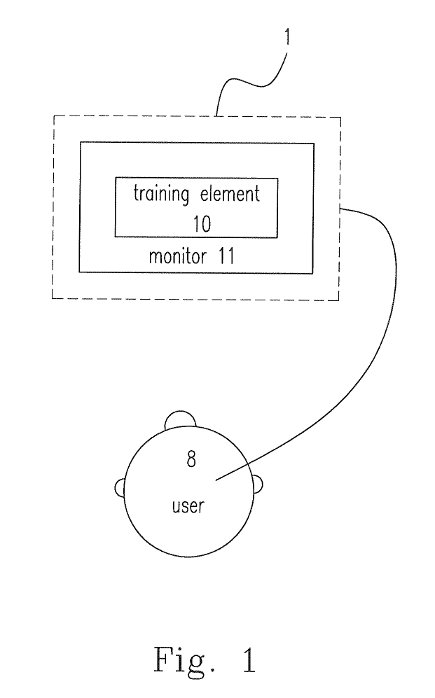

Please refer to FIG. 1, which is a diagram showing an embodiment of the apparatus of enhancing at least one of memory ability and parasympathetic activity of a subject of the present invention. The apparatus 1 mainly includes a training element 10 for increasing a mu rhythm of a user 8 of the neurofeedback training device 1.

According to the mentioned embodiment, for example, the training element 10 may be an animation displayed in a monitor 11. During the neurofeedback training process of the present invention, the animation is also an indicator indicating either an increase or a reducing of the mu rhythm of the user 8. Therefore, the user 8 will perform a neurofeedback training with an operant conditioning type by seeing the change of the animation, so as to successfully induce the mu rhythm by her/him self.

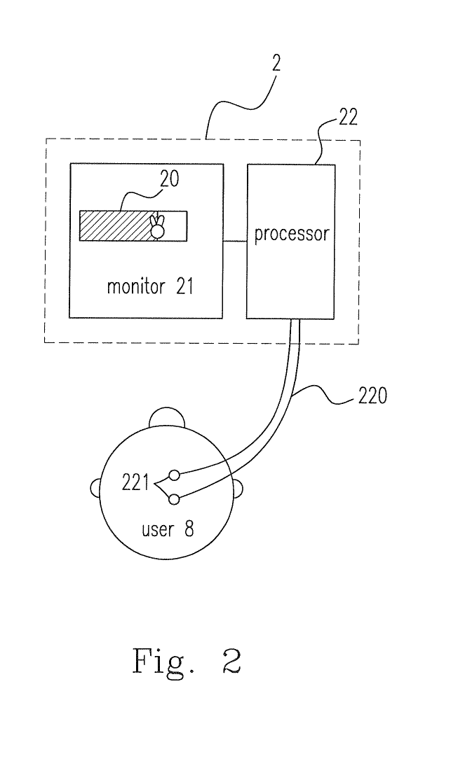

Please refer to FIG. 2, which is a diagram showing another embodiment of the apparatus of enhancing at least one of memory ability and parasympathetic activity of a subject of the present invention. The apparatus 2 includes a mu rhythm training interface 20, a monitor 21 and a processor 22. The processor 22 receives and processes a signal relevant to a mu rhythm, the monitor 21 is electronically connected to the processor 22, and the mu rhythm training interface 20 is displayed on the monitor 21.

According to the mentioned embodiment, the processor can include an electroencephalograph. The electroencephalograph may include a sensor 220 contacting with the brain of the user 8 and sensing the mu rhythm generated from the brain of the user 8. The sensor 220 may include at least a pair of electrode pads 221, which applies a signal capturing method with mutual-subtraction of signals of two electrodes to avoid interference of unnecessary noise. Furthermore, the processor 22 receives the signal relevant to the mu rhythm from a parietal lobe of the brain of the user 8, and the mu rhythm has a frequency ranged between 8-12 Hz.

In the embodiment shown in FIG. 2, only one pair of electrode pads 221 is attached to the parietal lobe of the brain of the user 8. However, in a practical application, more electrode pads, e.g. three pairs of electrode pads, may be used to collect several signals, and an average value thereof is selected, so as to increase the accurency of the received signal.

According to the mentioned embodiment, the mu rhythm training interface 20 may include an animation, which has the functions and operation ways similar to the training element 10 illustrated in FIG. 1. Furthermore, the monitor 21 includes one of a computer monitor and a cell phone monitor, and the apparatus 2 is one of a stationary device and a portable device. For example, the mu rhythm training interface 20 may be configured in a cell phone or a notebook connected to the processor 22, and thus a user may be trained anytime and anywhere.

According to the mentioned embodiments, the increasing of the mu rhythm of the user 8 includes the increasing of at least one of an energy and a lasting time period of the mu rhythm.

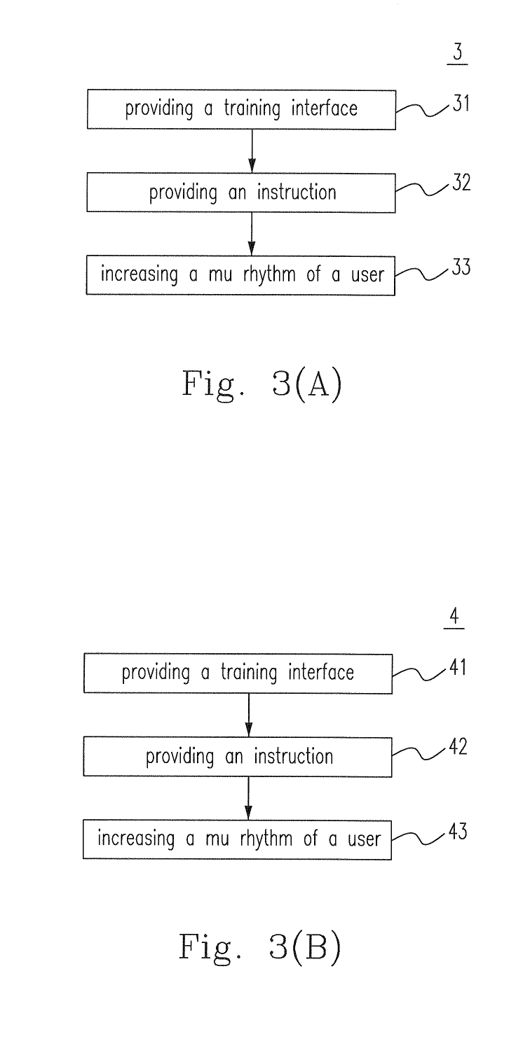

Please refer to FIG. 3(A), which is a flow chart showing an embodiment of a method of enhancing a memory ability of the present invention. The method 3 includes a step of increasing a mu rhythm of a user by using the training interface (step 33).

According to the mentioned embodiment, the step 33 includes the increasing of at least one of an energy and a lasting time period of the mu rhythm.

According to the mentioned embodiment, the method 3 may further include steps of providing a training interface (step 31) and providing an instruction for instructing the user in using the training interface to increase the mu rhythm (step 32). In the step 32, various instructions expected to facilitate the generation or increasing of the mu rhythm can be provided, e.g. an instruction teaching the user to relax her/his body. It shall be noted that the method of increasing a mu rhythm is not limited to the steps 31 and 32. After the training provided in the present application, the user may increase the mu rhythm by her/himself due to the training experience thereof.

According to the mentioned embodiment, the method 3 is an operant conditioning method, which is a conditioning method of learning to control the generation of signals. The principle of conditioning learning was first discovered by a psychologist, Edward Thorndike, in the early nineteenth century. Thorndike observed the action and strategy of a cat escaping out of a puzzle box, and provided the theory of law of effect, which stated that successful behaviors lead to satisfying outcomes and such successful experiences would be impressively remembered so that the successful behaviors will increase, whereas unsuccessful behaviors lead to disfavor feelings and such fail experiences would be discarded so that the frequency of unsuccessful behaviors will reduce. Briefly, successful behaviors would be likely to be repeated, whereas unsuccessful behaviors would be less likely to recur. In half of the nineteenth century, another psychologist, B. F. Skinner, provided principles of operant conditioning with core tools of reinforcement, punishment and extinction based on the theory of law of effect. In a neurofeedback training process, “feedback” is the “reinforcement” consequence in the principles of operant conditioning, which directly acts on central nervous system and is a way of directly affecting the body by the brain.

Please refer to FIG. 3(B), which is a flow chart showing an embodiment of a method of enhancing parasympathetic activity of the present invention. The method 4 includes a step of increasing a mu rhythm of a user by using the training interface (step 43).

According to the mentioned embodiment, the step 43 includes the increasing of at least one of an energy and a lasting time period of the mu rhythm.

According to the mentioned embodiment, the method 4 may further include steps of providing a training interface (step 41) and providing an instruction for instructing the user in using the training interface to increase the mu rhythm (step 42). In the step 42, various instructions expected to facilitate the generation or increasing of the mu rhythm can be provided, e.g. an instruction teaching the user to relax her/his body. It shall be noted that the method of increasing a mu rhythm is not limited to the steps 41 and 42. The method 4 is also an operant conditioning method. Therefore, after the training provided in the present application, the user may increase the mu rhythm by her/himself due to the training experience thereof.

The detailed experiments and drawings are provided in the following descriptions to verify the effects of improving human cognitive functions by the neurofeedback training device and method of the present invention.

In the prior researches on SMR and cognitive behavior, the obtained consequences are inconsistent. In some researches, it is found that the cognitive functions are improved when the energy of SMR signal is increased, but in some of other researches, it is found that the cognitive functions are improved while the energy of SMR signal is not increased. Therefore, two signals, i.e. high frequency SMR and mu rhythm, generated from different locations of the somatosensory cortex of the brain and having different functions and generation mechanisms, are compared in the experiments provided in the present application. Furthermore, results of the two experimental groups (SMR and Mu) are also compared with a control group having random signals.

Subjects

The present experiments are examined and allowed by the Institutional Review Board of Taiwan Cheng Kung University Hospital. There are 53 subjects, 22 male subjects and 31 female subjects, accomplishing the experiments, and the ages thereof are ranged between 18-29 years old. The 53 subjects are randomly divided into the three groups, in which the control group and the Mu group respectively have 18 people and the SMR group has 17 people.

Experimental Instruments

I. Brain and Physiology Signals

In the present experiments, an electrode cap sold by Neuroscan is adopted to perform brain waves measurements, and IBM x32 notebook is used together with a four channels signal amplifier, developed by the inventors and including three EEG amplifiers and one ECG, to record brain waves and physiology signals. The amplified signals pass through DAQ-6024E Analog/Digital Converter and connection block CB-68LP produced by National Instrument to be converted into digital signals, and the brain wave in each second is captured. The change of electric potential vs. time of the brain wave is converted to the change of power spectrum by fast Fourier transform, and the total power of the frequency band required by the respective groups (Mu: 8-12 Hz; SMR: 12-15 Hz; Control: 7-20 Hz) is calculated and instantly displayed on the monitor via a red histogram with a cartoon rabbit, so that the subject can be informed the current brain wave energy thereof for the observation and comparison. The red histogram with the cartoon rabbit is projected to a 20 inches liquid crystal display for the subject to perform the neurofeedback training.

II. Testing of Cognitive Behaviors

In the present experiments, the testing of cognitive behaviors applies psychology experimental software E-prime 2.0 executed in a 14 inches notebook, ASUS F6VE, and the evaluation of the abilities of cognitive behaviors, such as backward digit span, operation span and word-pair task, is performed.

Experimental Methods

I. Brain Wave Signals (Electroencephalograph, EEG)

The brain waves from three locations in the parietal lobe of the brain of the subject are recorded. The electrodes are attached to the areas in front and back of the three locations, having a distance about 2.5 cm therefrom, respectively, for recording signals. For obtaining better signals, mutual-subtraction of signals of two electrodes is adopted to avoid interference of unnecessary noise. A ground electrode is connected to the back of the right ear of the subject.

The original signals are instantly converted to magnitude of frequency energy by power spectrum analysis so as to perform the neurofeedback training. The energy frequency bands are divided into three groups, i.e. Mu (8-12 Hz), SMR (12-15 Hz) and random signal (7-20 Hz) according to the feedback signals. In the groups of Mu and SMR, the feedback signal is the domain energy in the designated frequency bands of 8-12 Hz and 12-15 Hz, respectively. In the group of random signal, four lengths are randomly selected as the feedback signal, e.g. 11-15, 16-20, 15-19 and 7-10 Hz. The subjects are randomly divided into the mentioned groups to perform the training.

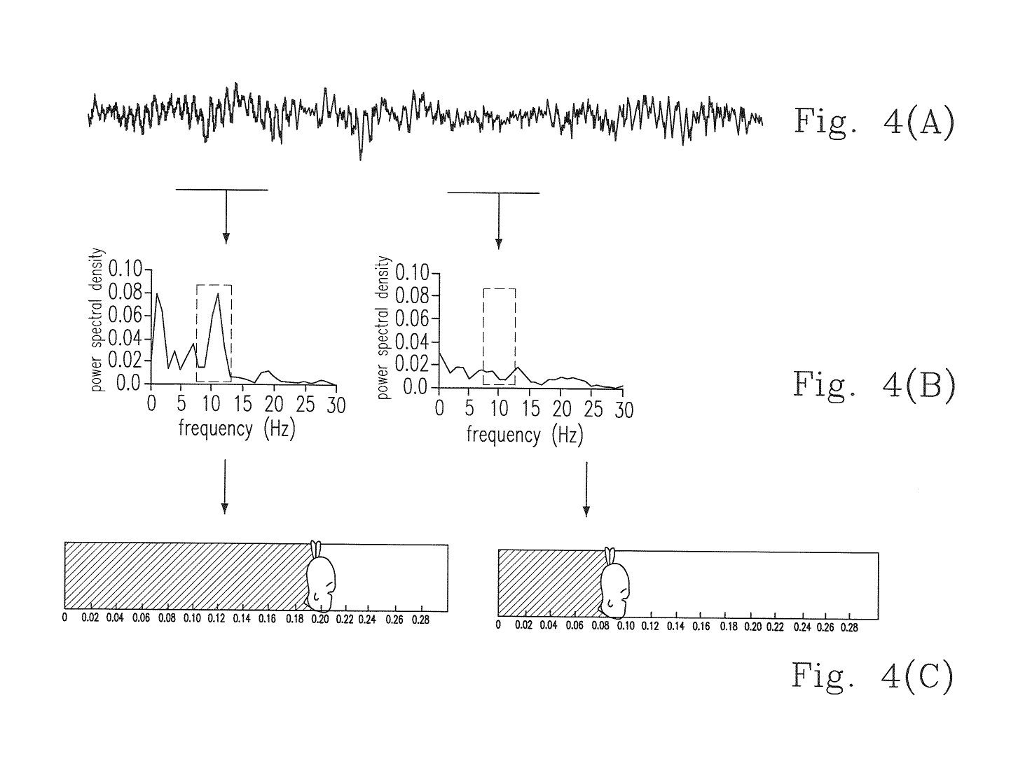

Please refer to FIGS. 4(A) to 4(C), which are diagrams showing the ways of recording brain wave and displaying training interface in the present invention. FIG. 4(A) shows the change of activity of brain potential along a time line. The power spectrum in different brain wave domains can be calculated and shown in FIG. 4(B). For mu rhythm, the total amount of the power in the frequency band of 8-12 Hz is calculated and shown as FIG. 4(C) with an animation, in which a cartoon rabbit moves forward or backward according to the magnitude of the total amount of the power. Therefore, the subject will instantly see the energy of the brain wave of 8-12 Hz. In FIGS. 4(A) to 4(C), the brain wave in the left domain has a clear peak in 8-12 Hz and the total power thereof is larger than a threshold value, whereas the brain wave in the right domain does not have a clear peak in 8-12 Hz and the total power thereof is smaller than a threshold value.

II. Physiological Signals (Electrocardiograph, EEG)

Periodic potential change of heart can be observed by the measurement of EEG. Signals of heartbeat are measured by using ECG electrode patches attached to the second and third ribs in the right side and the second rib, counted backwards, in the left side of the subject.

Experimental Processes

The subject is allowed to understand necessary processes of the experiments before participating therein, and the instructions are explained thereto during the neurofeedback training. The experimental processes are divided to three stages: before the neurofeedback training (Pretest), one-month neurofeedback training, and after the neurofeedback training (Posttest).

The cognitive abilities of the subject is tested and evaluated before and after the neurofeedback training. The evaluation of the cognitive abilities includes word-pair test, backward digit span test and operation span test.

Before and after one-month neurofeedback training and on each day during the neurofeedback training, the physiological signals of the subject shall be recorded by ECG, so that the change of autonomic nervous activity can be objectively recorded. Autonomic nervous activity can be objectively obtained from the analysis of heart rate variability (HRV), and is highly relevant to the emotion, dysphoria and blues of the subject. Therefore, the indexes calculated by using the analysis of HRV will facilitate the observation of the change of autonomic nervous activity of the subject everyday.

During the neurofeedback training, the subject is allowed to sit on a chair in a relaxed station and look at the monitor to perform a measurement (2 minutes) of the baseline of brain electronic potential activity, which is used to be a threshold of the analysis of power spectrum afterward. Subsequently, the neurofeedback training is performed six times (6 minutes each time), and the total time including the break time is 45 minutes. Finally, after the end of the neurofeedback training, an analysis of HRV is performed once (6 minutes).

Analysis Methods

I. Brain Wave Signals

After recording the original signals, the effects of interference signals from heartbeat are dealt with by Independent-component-analysis (ICA), then a power spectrum conversion is performed for the signals by Fourier analysis to convert the signals without heartbeat interference into mu rhythm (8-12 Hz) and SMR (12-15 Hz), and then the artifact of the subject is excluded. A value larger than 1.5 times of the threshold is defined as a generation of signals. The signal values are summed and divided by 1.5 times of the threshold to determine whether a signal is generated, since the threshold has an influence on the magnitude of an initial signal. Three groups of signals are compared by using two-way repeat measure ANOVA to determine whether a significant difference exists therein.

II. Analysis of HRV

The physical and mental state, which is corresponding to the autonomic nervous activity, of the subject can be evaluated based on the analysis of HRV. The calculation method is mainly to analyze time sequence between two heartbeats obtained by ECG or pulse measurement. The analysis of HRV mainly includes time domain analysis and frequency domain analysis.

QRS wave of heartbeat is read and determined by using Labview program, and the analysis of HRV is performed by using Kubio software, which provides time domain, frequency domain and non-linear HRV analyzing values at the same time.

III. Analysis of Cognitive Ability Tasks

By using two-way repeat measure ANOVA, the accuracy and response time of backward digit span test and word-pair test are analyzed, respectively, and the response time of operation span test is analyzed.

Experimental Results

I. Analysis Results of Brain Wave Signals

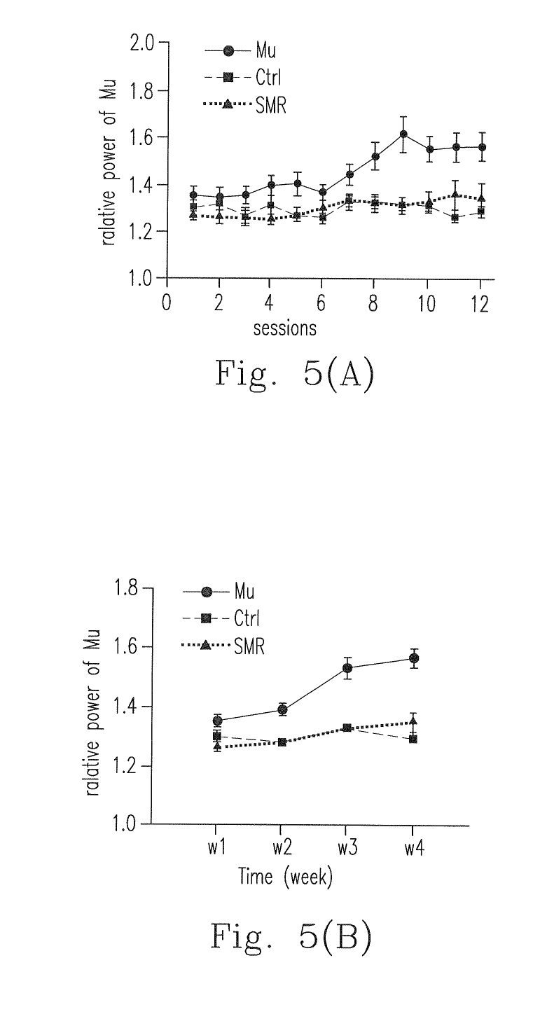

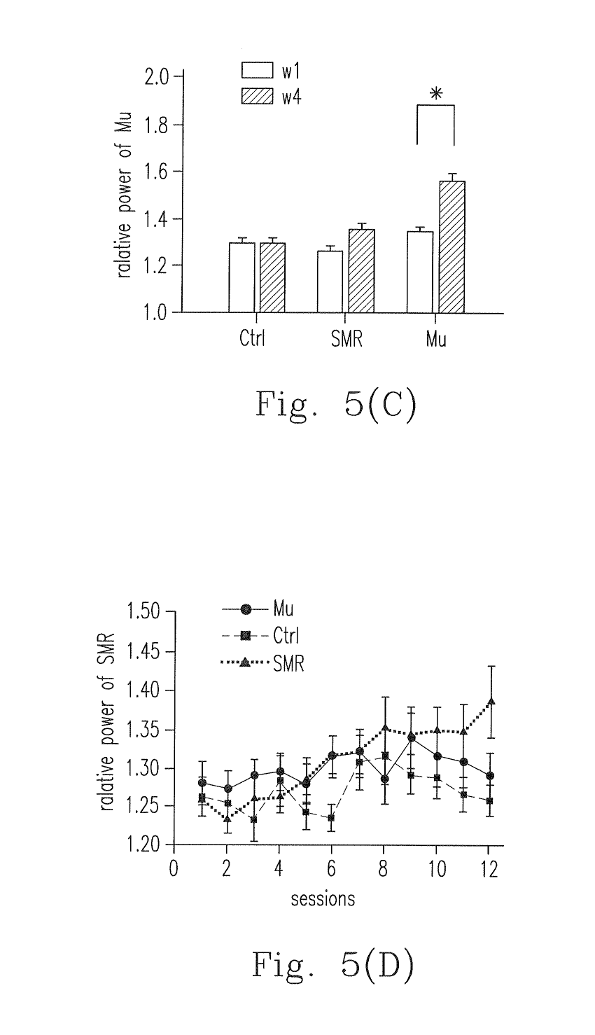

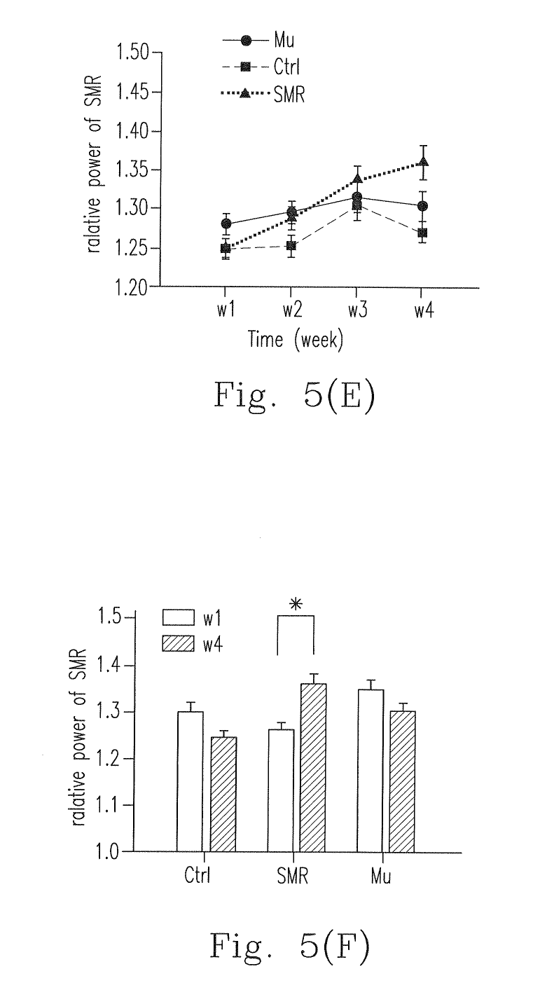

Please refer to FIGS. 5(A) to 5(F), which are diagrams showing change of energy of Control, SMR and Mu groups, respectively, during the neurofeedback training. In FIGS. 5(A), 5(B), 5(D) and 5(E), the black solid line with round dots represents Mu group, the black broken line with square dots represents Control group (Ctrl), the black dotted line with triangular dots represents SMR group, Y axis represents relative power, and X axis represents time.

In FIG. 5(A), whether the energy occurs or not is determined by a mu power larger than 1.5 times of the threshold. The determined value is divided by an average of the threshold values in two minutes to present the change of energy of Control, SMR and Mu groups in twelve trainings. It has been shown in FIG. 5(A) that mu power of Mu group, compared with those of the remaining two groups, is towards increasing after the trainings.

In FIG. 5(B), times of trainings are divided by weeks, which are divided to the first week (w1), the second week (w2), the third week (w3) and the fourth week (w4). It can be found that the change of mu power of Mu group has apparent differences in the third and fourth weeks.

In FIG. 5(C), the white bar represents the change of mu power in the first week of training, and the twill bar represents that in the fourth week. The mu power of Mu group has significant increasing between the first and fourth weeks (p<0.05), whereas the other two groups have no significant increasing.

In FIG. 5(D), SMR power is divided by an average of the threshold values in two minutes to present the change of energy of Control, SMR and Mu groups in twelve trainings, and only the value larger than 1.5 times of the threshold can be considered. It is found that SMR power of SMR group, compared with those of Mu and Control groups, is towards increasing after the trainings.

In FIG. 5(E), times of trainings for SMR power are also divided by weeks. It can be found that the change of SMR power of SMR group has apparent increasing trend in the fourth week, that of Mu group also has increasing trend, but that of Control group does not have such a trend.

In FIG. 5(F), the differences of SMR power between the first and fourth weeks of Control, SMR and Mu groups are compared. The SMR power of SMR group has significant increasing between the first and fourth weeks (p<0.05), whereas the other two groups have no significant increasing.

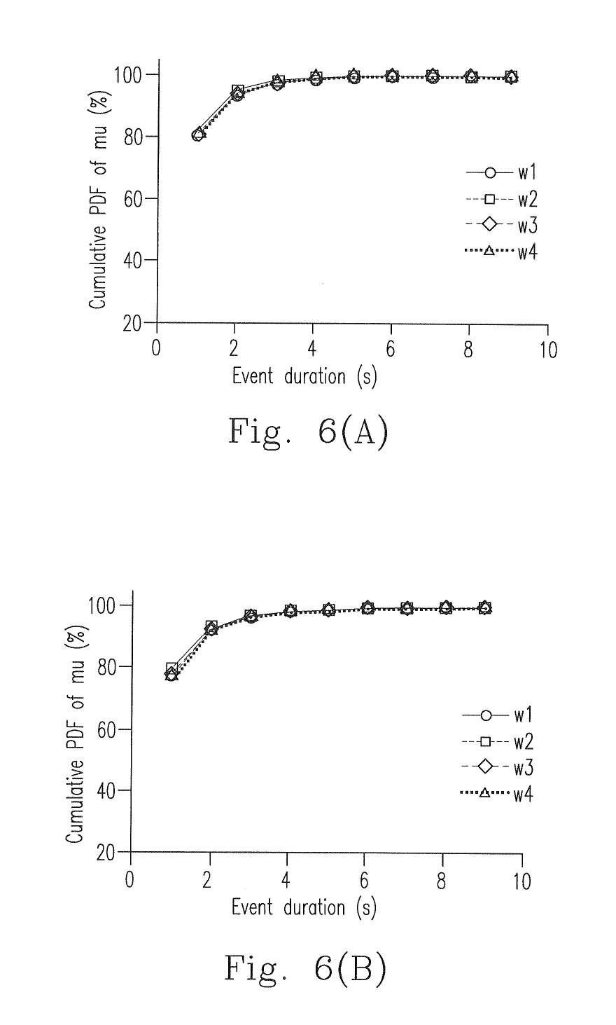

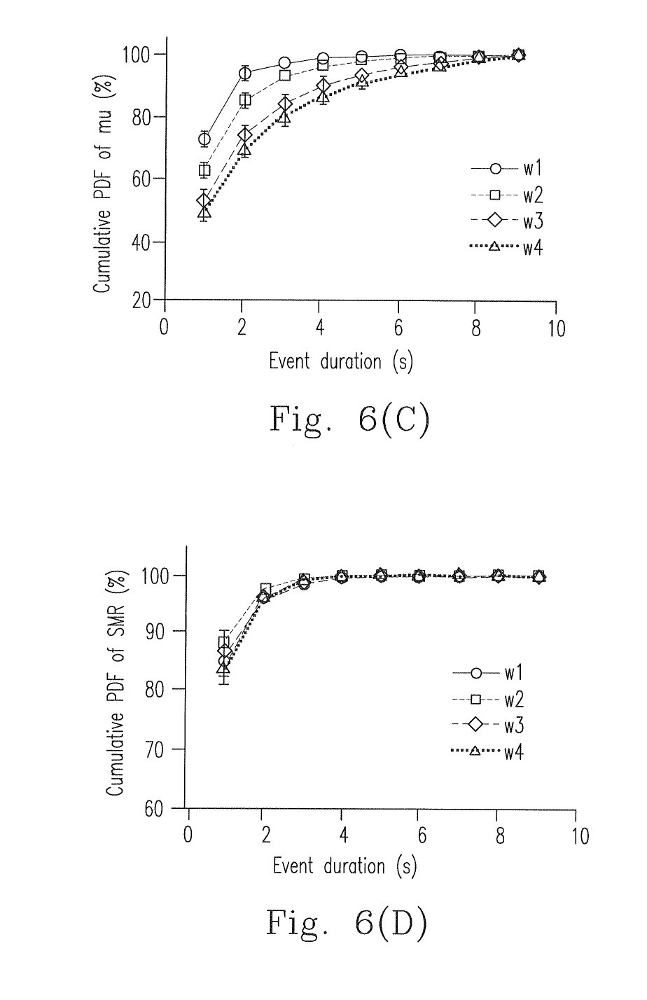

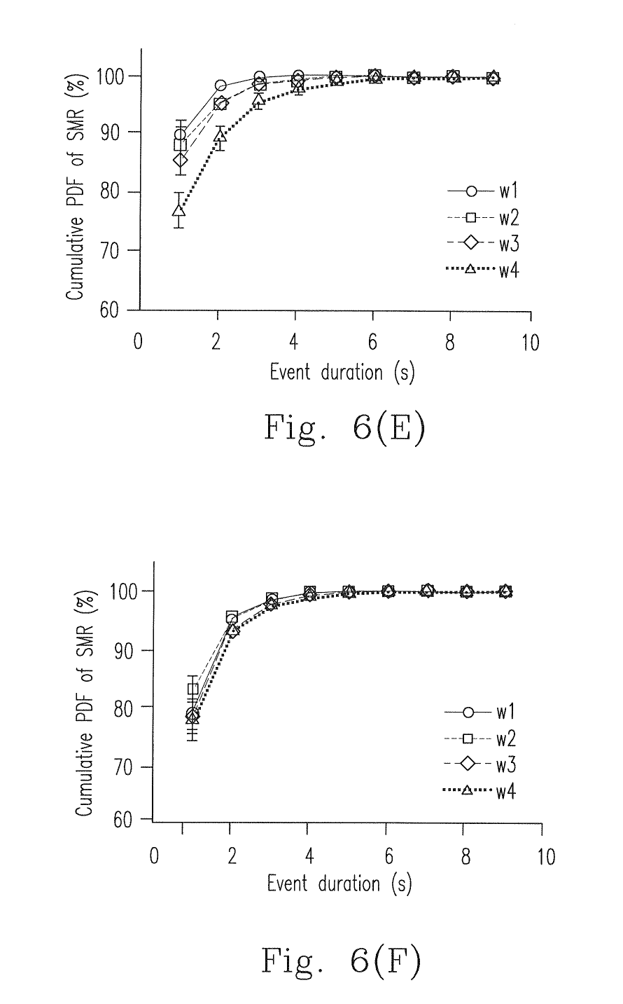

Please refer to FIGS. 6(A) to 6(F), which are cumulative length diagrams for the occurred signals of the respective groups. The cumulative ratio of occurrence length of signals are calculated, and the cumulative ratio is diagramed by the average with a unit of week for twelve trainings. The Y axis represents cumulative power length, and the X axis represents duration that the signal occurs. The solid line with round dots represents the first week, the broken line with square dots represents the second week, the broken line with rhombus dots represents the third week, and the dotted line with triangular dots represents the fourth week.

FIGS. 6(A), 6(B) and 6(C) show the performance of occurrence length of mu rhythm for Control, SMR and Mu groups, respectively. FIGS. 6(D), 6(E) and 6(F) show the performance of occurrence length of SMR signals for Control, SMR and Mu groups, respectively.

In FIGS. 6(A) and 6(D) for Control group, it shows that the frequency of occurrence length of signals at different training timepoints is about 1 to 2 seconds, and there is no difference among different training timepoints.

FIG. 6(B) shows that the performance of occurrence length of mu rhythm of SMR group is similar to that of Control group, while FIG. 6(E) shows that in SMR group, the length of SMR signals is longer and longer at different training timepoints and has an increasing at 3 to 4 seconds of the fourth week.

FIG. 6(C) shows that in Mu group, the occurrence length of mu signal at different training timepoints is longer and longer, while FIG. 6(F) shows that length of SMR signal does not have apparent increasing.

II. Analysis Results of HRV

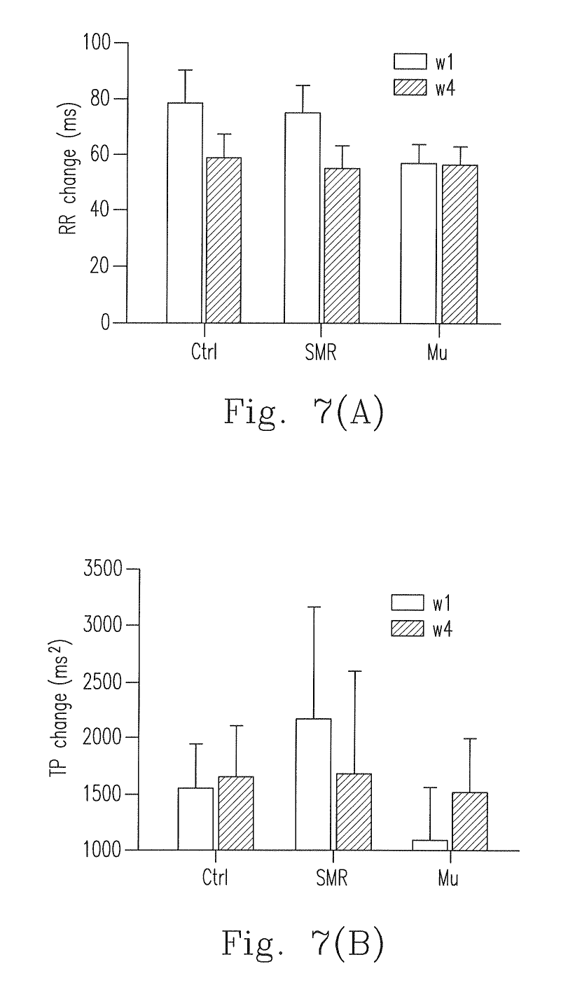

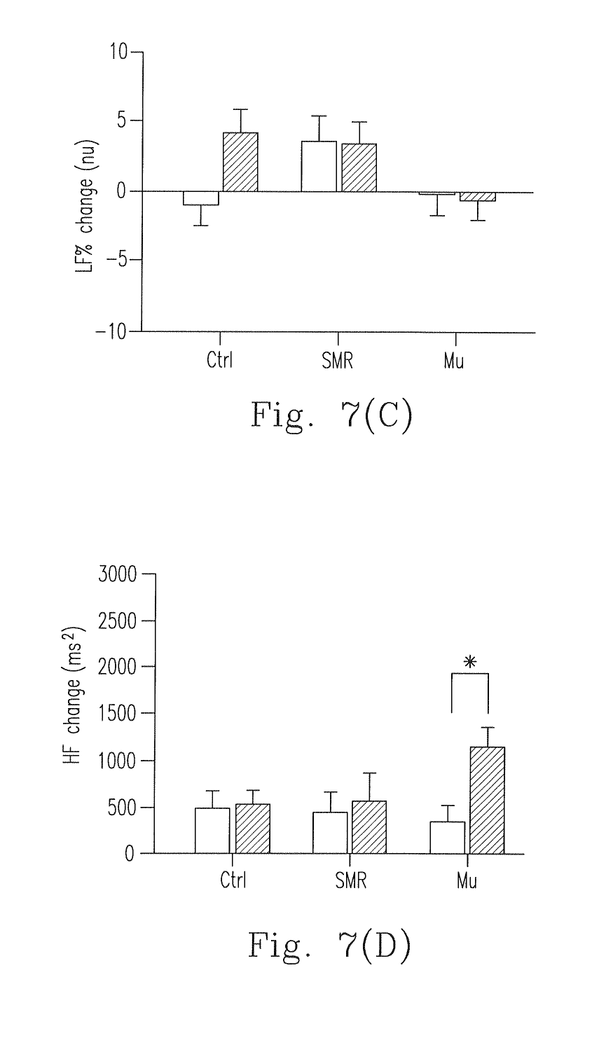

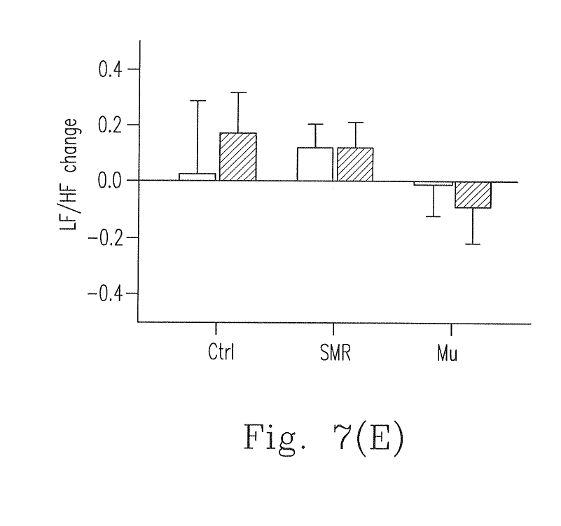

Please refer to FIGS. 7(A) to 7(E), which are contrast diagrams showing varient amounts of the analysis of HRV for Control, SMR and Mu groups. Five HRV indexes, i.e. RR interval, total power (TP) 0.01 to 0.4 Hz, low-frequency power (LF), high-frequency power (HF), and LF/HF ratio, are used to compare the difference between pretest (w1) and posttest (w4) of the respective groups.

FIG. 7(A) shows that RR values of the three groups do not have statistically significant difference between pretest and posttest. FIG. 7(B) shows that TP values of the three groups do not have statistically significant difference between pretest and posttest. FIG. 7(C) shows that the change of LF does not have significant difference in the respective groups. FIG. 7(D) shows that HF change between pretest and posttest in Mu group has significant difference (p<0.05), and HF is relative to parasympathetic activity. FIG. 7(E) shows that LF/HF value between pretest and posttest in Mu group is significantly decreasing.

III. Results of Evaluation of Cognitive Abilities

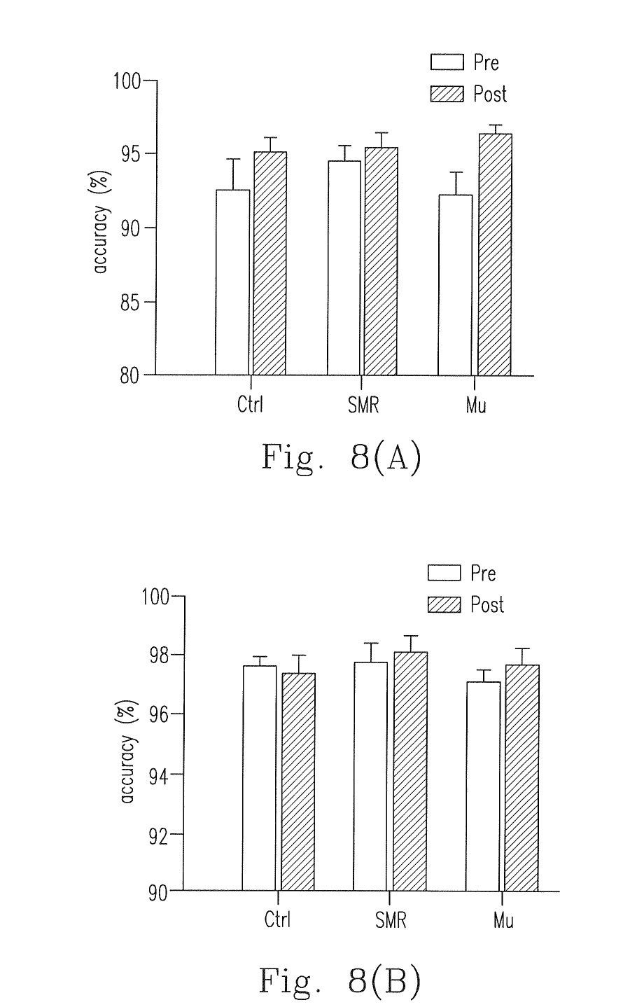

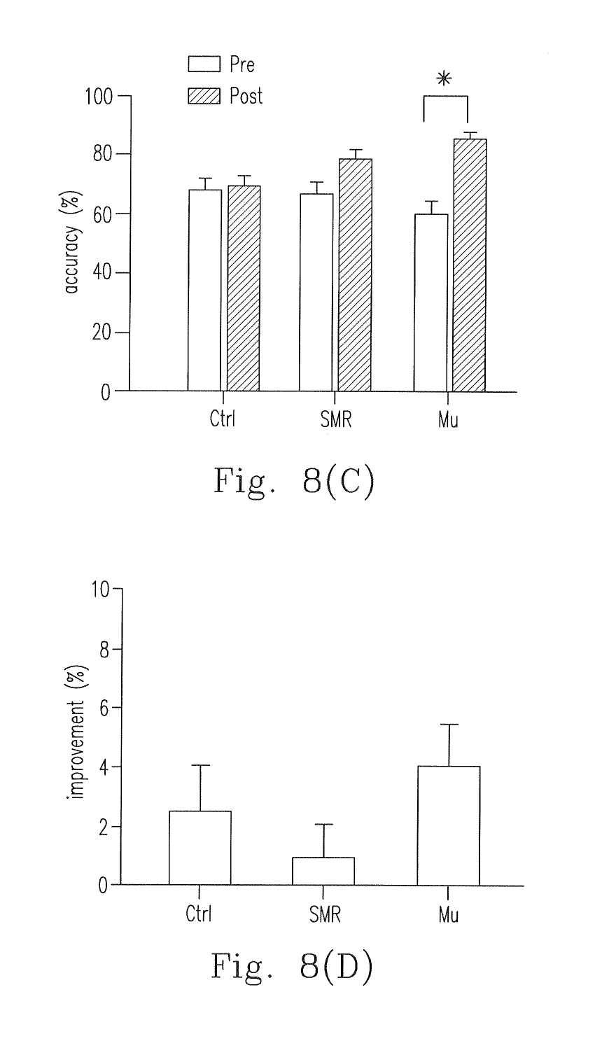

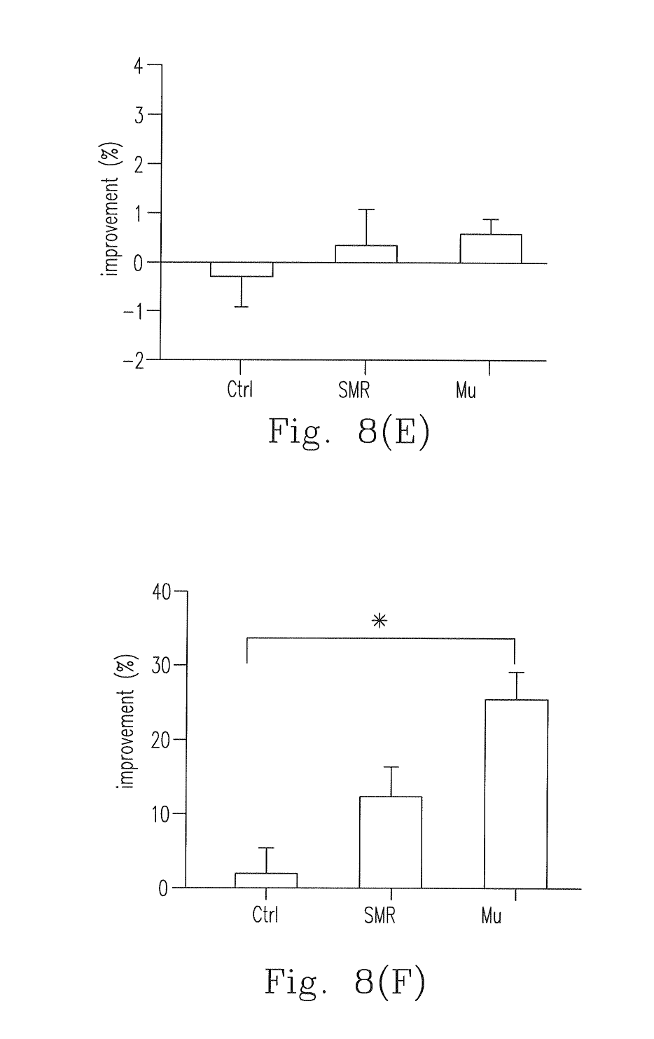

Please refer to FIGS. 8(A) to 8(F), which are contrast diagrams showing the accuracy and difference value of the evaluation of cognitive ability for the respective groups. In FIGS. 8(A), 8(B) and 8(C), the Y axis represents accuracy rate, and in FIGS. 8(D), 8(E) and 8(F), the Y axis represents improvement rate.

FIG. 8(A) shows performance of accuracy of backward digit span test for the respective groups between pretest and posttest. It can be found that the performance of posttest is better than that of pretest in each group, though the values of the three groups do not have statistical difference.

FIG. 8(B) shows performance of accuracy of operation span test for the respective groups. It can be found that the performance of posttest is better than that of pretest in both SMR and Mu groups, though the values of the three groups do not have statistical difference.

FIG. 8(C) shows performance of accuracy of word-pair test for the respective groups. The values between pretest and posttest have statistical difference (F=37.517, p<0.001), and the performance of accuracy in SMR and Mu groups has statistical difference (p<0.05), respectively.

FIG. 8(D) shows performance of improvement of backward digit span test for the respective groups between pretest and posttest. It can be found that the performance of Mu group in backward digit span test has approximately significant difference.

FIG. 8(E) shows that the improvement rate of the respective groups in operation span test does not have statistical difference (F=0.639, p=0.536).

FIG. 8(F) shows that the improvement rate of the respective groups in word-pair test has statistically significant difference (F=10.375, p<0.001), and the improvement rate of Mu group in word-pair test is much higher than that of control or SMR groups.

For further verifying the correlation, the relevance among improvement variation in word-pair test, numbers of successful signal occurrence and successful signal power for Mu and SMR groups is discussed by Pearson product-moment correlation coefficient. Please refer to Table 1, Pearson product-moment correlations are performed for successful numbers (the difference of numbers of rhythm successfully occurring in six minutes), successful power (the difference of rhythm power in six minutes), and improvement variations of backward digit span (BDSI), operation span (OSI) and word-pair (WPI) of Mu and SMR groups, wherein the successful numbers and successful power are obtained by substhating those in the first training from those in the twelfth training. It can be found that in Mu group, variation of word-pair and successful numbers (γ=0.566, p<0.05) and successful signal power (γ=0.541, p<0.05) have statistically significant difference, whereas the results of Pearson product-moment correlation for SMR group show that the correlation between WPI and successful numbers, or that between WPI and successful signal power, is negative.

TABLE 1

| Variable | BDSI | OSI | WPI |
|---|---|---|---|
| Number of Mu group | 0.313 | 0.043 | 0.566* |
| Power of Mu group | 0.328 | 0.138 | 0.541* |
| Number of SMR group | 0.381 | 0.370 | −0.260 |
| Power of SMR group | 0.364 | 0.305 | −0.236 |

In the past researches, it has been found that the ability of word-pair is improved in SMR group, and so is in the experimental results of the present application. However, not only the performances of the respective groups in each test, but also the improvements are compared. The experimental results of the present application show that the improvement rates of Mu group are higher than those of the other two groups, and thus verify that mu rhythm is relative to the memory storing function of working memory. Furthermore, by discussing Pearson product-moment correlations, it is verified that for Mu group, the improvement variation of word-pair and numbers of successful signal, or successful signal power, have positive correlation. Accordingly, the experiments verify that mu rhythm and cognitive functions, particularly word-pair ability, have highly positive correlation.

Based on the above experiments and analysis, the following results may be concluded:

1. After the training by the neurofeedback training device and method, mu rhythm of subjects in Mu group and SMR of subjects in SMR group have significant increasing in power strength of signals, generation numbers of power and occurrence length of signals.

2. After the training, HRV indexes, e.g. HF and LF/HF, of the subjects in Mu group have significant changes.

3. After the training, the word-pair ability of the subjects in both Mu and SMR groups have significant improvements.

4. Mu group, when compared with control and SMR groups, the improvement variations of word-pair task have significant difference.

In the past researches, there is no consistent results of inducing SMR signal. In the present application, a determination standard of 1.5 times of threshold is established to determine whether a signal occurs, and SMR signals are successfully induced in this standard. Similarly, such a standard (1.5 times of threshold) is used to determine whether mu rhythm occurs, and mu rhythm is also successfully induced in this standard. Therefore, both SMR and mu rhythm can be successfully induced by the neurofeedback training device and method of the present application.

Furthermore, the experimental results of the present application show that after the traing of increasing mu rhythm, HF relative to parasympathetic activity is successfully increased, while there is no such a variation in control and SMR groups after training. In the balance of sympathetic/parasympathetic, LF/HF is significantly reduced, while there is no such a variation in the other groups. Therefore, the mentioned indexes verify that the inducing of mu rhythm can directly affect the relaxation and activation of autonomic nervous system, and apparently indicate that mu rhythm will activate parasympathetic.

As to the evaluation of cognitive abilities, backward digit span task is relative to storing, monitoring and converting abilities, operation span task is relative to correlation of vocabulary and operation abilities, and word-pair task is relative to memory storing ability. The mentioned three tasks are performed to evaluate different cognitive functions. The evaluation of cognitive abilities in the present application is not a simple test for essential attention, whereas working memory to be converted by cognitive functions is necessary therefore, and accuracy is an essential reference. Though the accuracy of backward digit span task and operation span task, respectively, has no significant difference for the three groups, Mu group has significant improvement in word-pair task and it has been verified that mu rhythm and cognitive function of word-pair ability have highly positive correlation.

### Embodiments

1. An apparatus of enhancing at least one of a memory ability and a parasympathetic activity of a subject, comprising a training element for increasing a mu rhythm of the subject.

2. An apparatus of embodiment 1, further comprising a processor receiving and processing a signal relevant to the mu rhythm, and a display device electrically connected to the processor and displaying the training element thereon.

3. An apparatus of any of the preceding embodiments, wherein the processor receives the signal from a parietal lobe of the subject.

4. An apparatus of any of the preceding embodiments, wherein the display device includes one of a computer monitor and a cell phone display panel.

5. An apparatus of any of the preceding embodiments, being one of a stationary apparatus and a portable apparatus.

6. An apparatus of any of the preceding embodiments, wherein the mu rhythm has a frequency ranged between 8-12 Hz.

7. An apparatus of any of the preceding embodiments, wherein the training element includes an animation.

8. A method of enhancing a memory ability of a subject, comprising a step of increasing a mu rhythm of the subject.

9. A method of embodiment 8, wherein the step of increasing the mu rhythm includes increasing at least one of an energy and a lasting time period of the mu rhythm.

10. A method of any of the preceding embodiments, further comprising a step of providing a training interface for increasing the mu rhythm.

11. A method of any of the preceding embodiments, further comprising a step of providing an instruction for instructing the subject in using the training interface to increase the mu rhythm.

12. A method of any of the preceding embodiments, wherein the training interface includes an animation.

13. A method of any of the preceding embodiments, being an operant conditioning method.

14. A method of any of the preceding embodiments, wherein the mu rhythm has a frequency ranged between 8-12 Hz.

15. A method of enhancing a parasympathetic activity of a subject, comprising a step of increasing a mu rhythm of the subject.

16. A method of embodiment 15, wherein the step of increasing the mu rhythm includes increasing at least one of an energy and a lasting time period of the mu rhythm.

17. A method of any of the preceding embodiments, further comprising a step of providing a training interface for increasing the mu rhythm.

18. A method of any of the preceding embodiments, further comprising a step of providing an instruction for instructing the subject in using the training interface to increase the mu rhythm.

19. A method of any of the preceding embodiments, being an operant conditioning method.

20. A method of any of the preceding embodiments, wherein the mu rhythm has a frequency ranged between 8-12 Hz.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. An apparatus of enhancing at least one of a memory ability and a parasympathetic activity of a subject, comprising:
a training element for increasing a mu rhythm of the subject.

2. An apparatus as claimed in claim 1, further comprising:
a processor receiving and processing a signal relevant to the mu rhythm; and
a display device electrically connected to the processor and displaying the training element thereon.

3. An apparatus as claimed in claim 2, wherein the processor receives the signal from a parietal lobe of the subject.

4. An apparatus as claimed in claim 2, wherein the display device includes one of a computer monitor and a cell phone display panel.

5. An apparatus as claimed in claim 1, being one of a stationary apparatus and a portable apparatus.

6. An apparatus as claimed in claim 1, wherein the mu rhythm has a frequency ranged between 8-12 Hz.

7. An apparatus as claimed in claim 1, wherein the training element includes an animation.

8. A method of enhancing a memory ability of a subject, comprising a step of:
increasing a mu rhythm of the subject.

9. A method as claimed in claim 8, wherein the step of increasing the mu rhythm includes increasing at least one of an energy and a lasting time period of the mu rhythm.

10. A method as claimed in claim 8, further comprising a step of providing a training interface for increasing the mu rhythm.

11. A method as claimed in claim 10, further comprising a step of providing an instruction for instructing the subject in using the training interface to increase the mu rhythm.

12. A method as claimed in claim 10, wherein the training interface includes an animation.

13. A method as claimed in claim 8, being an operant conditioning method.

14. A method as claimed in claim 8, wherein the mu rhythm has a frequency ranged between 8-12 Hz.

15. A method of enhancing a parasympathetic activity of a subject, comprising a step of:
increasing a mu rhythm of the subject.

16. A method as claimed in claim 15, wherein the step of increasing the mu rhythm includes increasing at least one of an energy and a lasting time period of the mu rhythm.

17. A method as claimed in claim 15, further comprising a step of providing a training interface for increasing the mu rhythm.

18. A method as claimed in claim 17, further comprising a step of providing an instruction for instructing the subject in using the training interface to increase the mu rhythm.

19. A method as claimed in claim 15, being an operant conditioning method.

20. A method as claimed in claim 15, wherein the mu rhythm has a frequency ranged between 8-12 Hz.
